(19) United States
(12) Patent Application Publication
(10) Pub. No.: US 2005/0123542 A1
(43) Pub. Date: Jun. 9, 2005

(54) Methods for treating disorders involving monocytes

(75) Inventors: Frank Beurskens, Utrecht (NL); Janine Schuurman, Amsterdam (NL); Paul Parren, Odyk (NL); Jorgen Petersen, Rungsted Kyst (DK); Ole Baadsgaard, Malmo (SE)

Correspondence Address: LAHIVE & COCKFIELD, LLP., 28 STATE STREET, BOSTON, MA, 02109, US

(73) Assignee: GENMAB A/S

(21) Appl. No.: 10/982,725

(22) Filed: Nov. 4, 2004

Related U.S. Application Data
(60) Provisional application No. 60/518,552, filed on Nov. 6, 2003.

(51) Int. Cl. A61K 39/395; C07K 16/24; A61K 38/17; A61K 38/20

(52) U.S. Cl. 424/145.1; 530/388.23; 514/12; 424/85.2

(57) ABSTRACT

Methods for treating disorders involving monocytic activity by administering IL-15 antagonists that induce apoptosis of monocytes are disclosed.

### RELATED APPLICATIONS

This application claims priority to U.S. Ser. No. 60/518,552 filed on Nov. 6, 2003, the content of which is incorporated herein by reference.

### BACKGROUND OF THE INVENTION

Monocytes are white blood cells which are derived from pro-monocytes, which are found in bone marrow. Pro-monocytes differentiate into monocytes which are released into the blood and circulate in the blood until they are attracted to a site of injury by the inflammation process. Once monocytes enter into tissues (such as spleen, liver, lung, and bone marrow tissues) they mature into macrophages (also referred to as mononuclear phagocytes). Macrophages are capable of engulfing and destroying foreign antigens as the main scavenger cells of the immune system.

An abnormal increase in the number of monocytes in the blood (monocytosis) can occur in response to chronic infections, autoimmune disorders, blood disorders, and cancers and can exacerbate such disorders. For example, an increase in the number of monocytes can lead to an overexpression of inflammatory cytokines expressed by monocytes which, in turn, perpetuate the inflammation and the cell-mediated immune response associated with monocytes.

IL-15 is a pro-inflammatory cytokine which is constitutively expressed on monocytes, as well as macrophages, fibroblasts, keratinocytes and dendritic cells (Waldmann and Tagaya (1999) Annu Rev Immunol 17:19-49; Fehniger and Caligiuri, (2001) Blood 97:14-28). IL-15 is known to mediate several immune responses, such as T cell proliferation, TNFα production and recruitment of immune cells. Accordingly, IL-15 activity has been implicated in the pathogenesis of several inflammatory diseases.

Accordingly, the need exists to develop selective methods for downregulating IL-15 activity and associated immune responses, particularly recruitment of monocytes, to treat inflammatory diseases.

### SUMMARY OF THE INVENTION

The present invention is based, in part, on the discovery that IL-15 antagonists can induce apoptosis of monocytes. The present invention provides a variety of new diseases that were previously not known to be treatable using IL-15 antagonists. Accordingly, in one embodiment, the invention provides a method for treating a disease involving monocytic activity comprising administering a therapeutically effective amount of an IL-15 antagonist that induces apoptosis of monocytes, wherein the disease is arthritides, connective disorders, neurological disorders, gastrointestinal and hepatic disorders, allergic disorders, hematologic disorders, skin disorders, pulmonary disorders, malignant disorders, endocrinological disorders, vasculitides, infectious disorders, kidney disorders, or muscle disorders. Additional disorders that can be treated include cardiac disorders, circulatory disorders, metabolic disorders, coagulation disorders, and bone disorders.

IL-15 antagonists encompassed by the present invention include a broad variety of molecules that antagonize or inhibit IL-15 activity (i.e., IL-15 mediated anti-apoptosis) including, but not limited to, anti-IL-15 antibodies, anti-IL-i 5R antibodies, soluble IL-15Rs, IL-15 muteins, anti-IL-15 small molecules and anti-IL-15R small molecules. Other antagonists, such as binding proteins and peptide mimetics, which are capable of inhibiting IL-15 activity, also are included. In a particular embodiment, the antagonist is capable of interfering with the assembly of the IL-15Rα, β, and γ subunits, e.g., the antagonist binds to an epitope located on the β- or γ-chain interacting domain of IL-15. In another particular embodiment, the antagonist is an IL-15 mutein, e.g., an IL-15 mutant that is capable of binding to IL-15Rα but is not able to bind to either or both of the β- and/or γ-subunits of IL-15R and, therefore, is not able to effect signaling.

In one embodiment, the IL-15 antagonist is an anti-IL-15 antibody, e.g., a human anti-IL-15 antibody, or an antibody fragment or a single chain antibody thereof, capable of inducing apoptosis of monocytes. Particular anti-IL-15 antibodies useful in the invention include those disclosed in WO 03/017935 (corresponding to US 2003/0138421).

In one embodiment, the IL-15 antagonist is a monoclonal human IgG1 anti-IL-15 antibody having heavy chain and kappa light chain variable regions which comprise the amino acid sequences shown in SEQ ID NO:2 and SEQ ID NO:4, respectively, mAb 146B7, or conservative sequence modifications thereof. In another embodiment, the antibody comprises at least one CDR sequence having the amino acid sequences shown in SEQ ID NOs:5, 6, 7, 8, 9, or 10, or conservative sequence modifications thereof which have 1 to 3 amino acid deletions, substitutions, or additions compared to SEQ ID NOs:5, 6, 7, 8, 9, and 10, or fragments thereof which retain the ability to specifically bind to human IL-15. In yet another embodiment, the antibody is encoded by human IgG heavy chain and human kappa light chain nucleic acids comprising nucleotide sequences in their variable regions as set forth in FIG. 1 (SEQ ID NO:1) and FIG. 2 (SEQ ID NO:3), respectively, or conservative sequence modifications thereof. In still another embodiment, the antibody is an IgG1 isotype and has a heavy chain variable region comprising the amino acid sequence shown in FIG. 1 (SEQ ID NO:2), or conservative sequence modifications thereof. A further embodiment includes an antibody which is an IgG1 isotype and has a kappa light chain variable region comprising the amino acid sequence shown in FIG. 2 (SEQ ID NO:4), respectively, or conservative sequence modifications thereof.

Moreover, depending on the disease, therapy with the IL-15 antagonist can be performed ex vivo or in vivo.

### BRIEF DESCRIPTION OF THE DRAWINGS

FIGS. 1a-f are graphs showing mAb 146B7 inhibits IL-15 mediated protection of apoptosis by IL-15. Human PBMC were incubated with IL-15 (5 ng/ml) in culture medium alone (▪) or in combination with 10 μg/ml mAb 146B7 (O) or with 10 μg/ml isotype control antibody (anti-KLH) (▴) for 5 days. Apoptosis was measured using annexin V conjugated with FITC and propidium iodide (PI). Live (a and d), early (b and e), and late (c and f) apoptotic cells were defined by annexin V-FITC^{neg}PI^{neg}, annexin V-FITC^{pos}PI^{neg}, and annexin V-FITC^{pos}PI^{pos} staining by flow cytometry, respectively. Representative data of three individual experiments with six different PBMC donors are shown. (a, b and c) show that mAb 146B7 inhibits IL-15-induced survival of monocytes, whereas (d, e and f) show that no significant differences in comparison were found in the fraction of live and apoptotic T cells. The asterisk *: P<05, mAb 146B7 vs. no antibody treatment. The double asterisk **: P<005, mAb 146B7 vs. no antibody treatment and mAb 146B7 vs. anti-KLH.

### DETAILED DESCRIPTION OF THE INVENTION

The present invention provides methods for treating a variety of disorders by inducing apoptosis (i.e., programmed cell death) of immune cells involved in such disorders. Particular immune cells include, for example, monocytes, T cells, B cells, neutrophils, mast cells, keratinocytes, NK T cells, and NK cells. In a particular embodiment, the present invention provides methods for treating disorders involving monocytic activity by inducing apoptosis of monocytes.

The term “monocyte” as used herein refers, collectively, to both circulating monocytes and to macrophages present in tissue. The term “monocytic activity” refers to physiological effects (e.g., immune responses) caused directly or indirectly by monocytes, such as the proinflammatory effects of cytokines expressed by monocytes and other humoral and/or cell-mediated immune response induced by such cytokines.

IL-15 is an important proinflammatory cytokine expressed by monocytes. The term “IL-15,” as used herein, refers also to IL-15 antigen, Interleukin 15, IL-15 ligand, IL-15 polypeptide, and any variants or isoforms of human IL-15 which are naturally expressed by cells. IL-15 is a cytokine of the four-helix bundle family which shares many biological activities with IL-2 due to common receptor components. The receptor of IL-15 (“IL-15R”) is composed of three subunits; IL-15 Rα, IL-2Rβ and IL-2Rγ. Therefore, the functional receptors for IL-2 and IL-15 consist of a private α-chain, which defines the binding specificity for IL-2 or IL-15, and shared IL-2 receptor β- and γ-chains. The γ-chain is also a signaling component of IL-4, IL-7, IL-9, and IL-21 receptors. Thus, the γ-chain is called the common γ or γ-c. Monocytic activity inhibited by the methods of the present invention includes the proinflammatory effects caused by IL-15 (i.e., any humoral or cell-mediated immune response induced by IL-15). These effects include, for example, production of TNFα and other inflammatory mediators, recruitment/proliferation of T-cells, and reversal of monocytic apoptosis. These proinflammatory effects are the result of IL-15/IL-15R signaling, e.g., based on the assembly of the IL-15Rα, β (IL-2Rβ), and γ (IL-2Rγ) subunits.

Therapies of the present invention employ IL-15 antagonists that induce apoptosis of monocytes. As used herein, the terms “IL-15 antagonist” and “IL-15 inhibitor” are used interchangeably and include any compound that interferes with IL-15 mediated signaling through IL-15R and induces apoptosis of monocytes. For example, the antagonist can interfere with assembly of the IL-15Rα, β, and γ subunits. In a particular embodiment, the antagonist binds to IL-15 or IL-15R and antagonizes or inhibits IL-15 mediated signaling through IL-15R. In another particular embodiment, the antagonist binds to an epitope located on the β- or γ-chain interacting domain of IL-15.

As used herein, the terms “antagonizes IL-15 activity” and “inhibits IL-15 activity” are used interchangeably and are intended to include any measurable decrease in IL-15 activity. Accordingly, IL-15 antagonists encompassed by the present invention include binding agents, such as anti-IL-15 antibodies, anti-IL-15R antibodies, soluble forms of IL-15R, soluble ligands for IL-15R, IL-15 and IL-15R muteins, and small molecules that bind to IL-15 or IL-15R and inhibit IL-15 mediated signaling.

IL-15 antagonists of the invention can be identified using a number of art recognized assays that measure IL-15 activity, including the ability of IL-15 to prevent or reverse apoptosis of monocytes. Such assays, which quantify apoptosis of monocytes (e.g., present in a biological sample taken from a subject), include assays that image apoptosis using labeled reagents, such as annexin, as described in WO 98/48699 and WO 02/080754. In brief, the antagonist can be incubated with monocytes in a sample for a suitable period of time. Apoptosis can then be measured by staining the cells with labeled (e.g., FITC-labeled) annexin which binds to markers, such as exposed phospholipids, present on cells that have undergone apoptosis.

In one embodiment, the IL-15 antagonist is an anti-IL-15 antibody, e.g., a human anti-IL-15 antibody. IL-15 antibodies of the invention include a variety of antibody isotypes, such as IgG1, IgG2, IgG3, IgG4, IgM, IgA1, IgA2, IgAsec, IgD, and IgE. Typically, they include IgG1 (e.g., IgG1k), IgG3 and IgM isotypes. The generation of human monoclonal antibodies against IL-15, e.g., human monoclonal antibodies derived from hybridoma 146B7, is described in WO 03/017935. The term “antibody” as referred to herein includes whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., IL-15). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Furthermore, the antigen-binding fragments include binding-domain immunoglobulin fusion proteins comprising (i) a binding domain polypeptide (such as a heavy chain variable region or a light chain variable region) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

The present invention also encompasses “conservative sequence modifications” of the sequences set forth in SEQ ID NOs: 1-31, i.e., nucleotide and amino acid sequence modifications which do not significantly affect or alter the binding characteristics of the antibody encoded by the nucleotide sequence or containing the amino acid sequence. Such conservative sequence modifications include nucleotide and amino acid substitutions, additions and deletions. Modifications can be introduced into SEQ ID NOs: 1-31 by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a human anti-IL-15 antibody is preferably replaced with another amino acid residue from the same side chain family.

Alternatively, in another embodiment, mutations can be introduced randomly along all or part of an anti-IL-15 antibody coding sequence, such as by saturation mutagenesis, and the resulting modified anti-IL-15 antibodies can be screened for binding activity.

Accordingly, antibodies encoded by the (heavy and light chain variable region) nucleotide sequences disclosed herein and/or containing the (heavy and light chain variable region) amino acid sequences disclosed herein (i.e., SEQ ID NOs: 1-31) include substantially similar antibodies encoded by or containing similar sequences which have been conservatively modified. Further discussion as to how such substantially similar antibodies can be generated based on the partial (i.e., heavy and light chain variable regions) sequences disclosed herein as SEQ ID Nos:1-31 is provided in WO 03/017935.

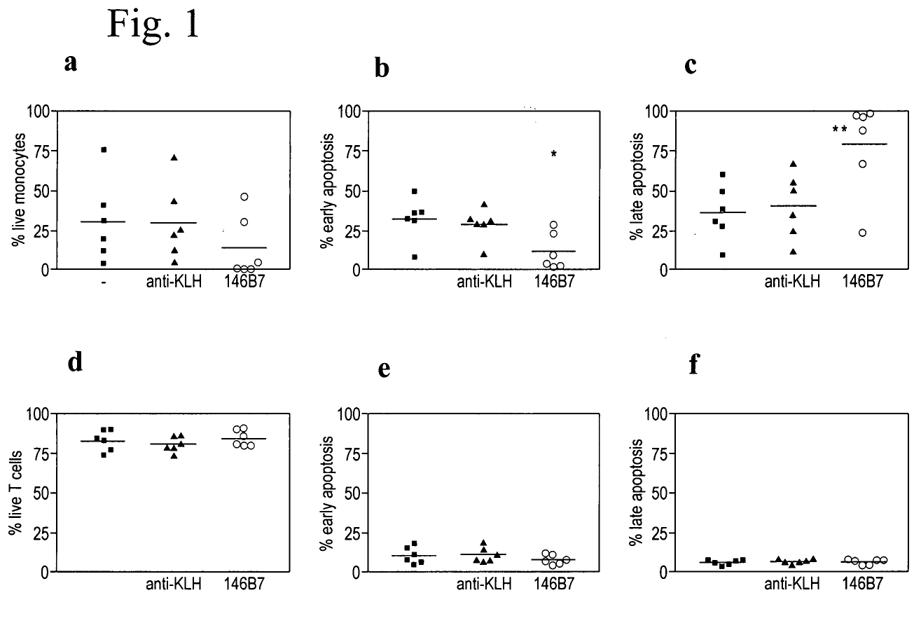

As exemplified herein, IL-15 antagonists, such as mAb 146B7, induce apoptosis of monocytes. In particular, Example 2 shows that mAb 146B7 reverses IL-15-mediated protection against apoptosis of monocytes (FIG. 1). Therefore, a wide variety of disorders mediated by monocytic activity can be treated using the methods of the present invention. As used herein, the term “inflammatory disease” includes a disease involving overexpression of inflammatory cytokines expressed by monocytes. In a particular embodiment, the invention includes methods of treating the diseases described in Table 1.

TABLE 1

| | |
|---|---|
| gout | |
| arthritides | |
| connective disorders | systemic sclerosis, retroperitoneal fibrosis |
| | familial Mediterranean fever, tumor |
| | necrosis factor receptor-associated |
| | periodic syndromes |
| neurological disorders | systemic sclerosis, stroke, cerebral trauma, |
| | Guillain-Barré syndrome/polytadiculitis, |
| | chronic inflammatory demyelinating |
| | polyneuropathy, and Alzheimer's disease |
| gastrointestinal and | alcoholic hepatitis, hepatitis C, acute |
| hepatic disorders | pancreatic, Whipple's disease, chronic |
| | active hepatitis, and sclerosing cholangitis |
| allergic disorders | chronic urticaria and angioedema |
| hematologic disorders | hemophagocytic syndrome and |
| | histiocytosis X |
| skin disorders | pemphigus vulgaris, toxic/irritative contact |
| | eczema, linear IgA dermatitis, dermatitis |
| | herpetiformis, epidermolysis bullosa |
| | acquisita, acne vulgaris and rosacea |
| pulmonary disorders | severe acute respiratory distress syndrome, |
| | pulmonary silicosis, berylliosis, and |
| | asbetosis |
| malignant disorders | prostatic cancer |
| endocrinological disorders | insulin-dependent diabetes mellitus, and |
| | subacute thyroiditis |
| vasculitides | panniculitis, erythema nodosum, and |
| | Behcet's syndrome |
| infectious disorders | leishmaniasis and infectious |
| | mononucleosis |
| kidney disorders | chronic renal failure, acute |
| | glomerulonephritis, chronic |
| | glomerulonephritis, ANCA-associated |
| | nephritides, and nephrosclerosis |
| cardiac disorders | acute myocardial infarction, acute |
| | coronary syndromes, and unstable |
| | coronary disease |
| circulatory disorders | arterial hypertension and pulmonary |
| | hypertension |
| metabolic disorders | Gaucher's disease and Fabry's disease |
| coagulation disorders | disseminated intravascular coagulation, |
| | thrombotic thrombocytopenic purpura, and |
| | hemolytic-uremic syndrome |
| bone disorders | osteoporosis |
| muscle disorders | muscle adipose disorder |

The IL-15 antagonists of the invention can be administered to a subject as a composition, e.g., a pharmaceutical composition, containing one or a combination of IL-15 antagonists, formulated together with a pharmaceutically acceptable carrier. In one embodiment, depending on the disease, therapy with the IL-15 antagonist can involve ex vivo procedures. For example, the subject can be administered cells which have been treated ex vivo to insert therein a DNA segment encoding an IL-15 antagonist. Upon administration to the subject, the treated cells express in vivo a therapeutically effective amount of the antagonist. As used herein, the term “subject” includes any human or nonhuman animal. The term “nonhuman animal” includes all vertebrates, e.g., mammals and non-mammals, such as nonhuman primates, sheep, dog, cow, chickens, amphibians, reptiles, etc. In a particular embodiment, the compositions include a combination of multiple (e.g., two or more) IL-15 antagonists wherein each of the antagonists of the composition inhibits different activities of IL-15.

IL-15 antagonists of the invention also can be administered as a combination therapy, e.g., combined with other agents that reduce monocytic activity and/or other immune activity. For example, the combination therapy can include an IL-15 antagonist and one or more additional therapeutic agents, such as anti-inflammatory agents, DMARDs (disease-modifying anti-rheumatic drugs), immunosuppressive agents, and chemotherapeutics. The IL-15 antagonists can also be administered in conjunction with radiation therapy. Further, the IL-15 antagonists can be co-administered with inhibitors of CD4 and/or IL-2, such as CD4-specific antibodies and IL-2 specific antibodies. Such combination therapies are known to be particularly useful for treating autoimmune diseases.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., the IL-15 antagonist, may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous acids and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

IL-15 antagonists can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Certain routes of administration may require coating the antagonist or inhibitor compound with, or co-administering the inhibitor compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions, such as compositions comprising IL-15 antagonists, typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the IL-15 antagonist may be administered once or twice weekly by subcutaneous injection or once or twice monthly by subcutaneous injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations encompassed by the methods of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of 100%, this amount will range from about 0.001% to about 90% of active ingredient, preferably from about 0.005% to about 70%, most preferably from about 0.01% to about 30%.

Formulations which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, the compounds containing the IL-15 antagonists may be used in a suitable hydrated form and/or formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular IL-15 antagonists employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required.

As used herein, the term “therapeutically effective amount” is understood to mean a nontoxic but sufficient amount, at dosages and for periods of time as necessary, to achieve the desired result. For example, an effective amount means the amount that results in the inhibition of IL-15 mediated protection of monocytes, i.e., the amount that results in apoptosis of monocytes in a subject. As described herein, assays which quantify apoptosis of monocytes (e.g., present in a biological sample taken from a subject), are known to those of ordinary skill in the art and include assays that image apoptosis using labeled reagents, such as annexin, as described in WO 98/48699 and WO 02/080754. For example, as shown in FIG. 1(a-c), a therapeutically effective amount of an IL-15 inhibitor includes an amount which is capable of increasing apoptosis, i.e., early apoptosis or late apoptosis, in a subject, when compared to a control, by at least about 5%, preferably at least about 8, 11, 14, 17, or 20%, more preferably at least about 23, 26, 29, 32, or 35%, more preferably at least about 36, 37, 38, 39, or 40%, more preferably at least about 41, 42, 43, 44, or 45%, more preferably at least about 50, 55, 60, 65, 70, or 75%. An increase in apoptosis of cells of at least about 80, 85, 90, 95, or 100% is also encompassed by the invention. Ranges intermediate to the above-recited values are also intended to be encompassed by the present invention.

An effective amount of an active compound, as defined herein, may vary according to factors such as the disease state, age, and weight of the subject, and the ability of the active compound to elicit a desired response in the subject. Dosage regimens may be adjusted to provide the optimum therapeutic response. An effective amount is also one in which any toxic or detrimental effects of the active compound are outweighed by the therapeutically beneficial effects. For example, the physician or veterinarian could start doses of the compounds employed in the pharmaceutical composition at levels lower than that required in order to achieve the desired therapeutic effect and gradually increase the dosage until the desired effect is achieved. In general, a suitable daily dose of a composition will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4.,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the IL-15 antagonists can be formulated to ensure proper distribution in vivo. For example, the blood-brain barrier (BBB) excludes many highly hydrophilic compounds. To ensure that the therapeutic compounds employed by the methods of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et aL); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds containing the IL-15 antagonists are formulated in liposomes; in a more preferred embodiment, the liposomes include a targeting moiety. In a most preferred embodiment, the therapeutic compounds in the liposomes are delivered by bolus injection to a site proximal to the tumor or infection. The composition must be fluid to the extent that easy syringability exists. It must be stable under the conditions of manufacture and storage and must be preserved against the contaminating action of microorganisms such as bacteria and fungi.

In a further embodiment, the IL-15 antagonists can be formulated to prevent or reduce the transport across the placenta. This can be done by methods known in the art, e.g., by PEGylation or by use of F(ab)2′ fragments. Further references can be made to “Cunningham-Rundles C, Zhuo Z, Griffith B, Keenan J. (1992) Biological activities of polyethylene-glycol immunoglobulin conjugates. Resistance to enzymatic degradation. J Immunol Methods. 152:177-190; and to “Landor M. (1995) Maternal-fetal transfer of immunoglobulins, Ann Allergy Asthma Immunol 74:279-283.

The ability of a compound to inhibit cancer can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

The ability of the IL-15 antagonists to treat or prevent other disorders involving monocytic activity can also be evaluated according to methods well known in the art.

The composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

The present invention is further illustrated by the following examples which should not be construed as further limiting.

### EXAMPLES

### Example 1

### Preparation of Anti-IL-15 Antibodies

Human monoclonal antibodies against IL-15 were obtained as described in US 2003/0138421.

### Example 2

### mAb 146B7 Induces Apoptosis of Monocytes in PBMC Culture

Culturing of peripheral blood mononuclear cells (PBMC): Blood was obtained by venipuncture from healthy volunteers after informed consent. Purification of PBMC was performed by density gradient centrifugation using Ficoll-Isopaque (Pharmacia, Uppsala, Sweden). PBMC were cultured in RPMI 1640 medium (Biowhittaker, Vervier, Belgium) supplemented with penicillin (50 U/ml), streptomycin (50 ng/ml), L-glutamine (2mM) (Biowhittaker Europe) and 10% fetal calf serum (Optimum C241, Multicell, Wisent, St. Bruno, Canada).

Apoptosis induction of PBMC by mAb 146B7: PBMC were cultured in 96-well U-bottom plates (Nalgene Nunc, Rochester, N.Y.), 1.5×10⁵ cells per well in the presence or absence of 5 ng/ml IL-15 (Immunex) and 10 μg/ml anti-IL-15 antibody (mAb 146B7) or 10 μ/ml human isotype control antibody (mAb anti-KLH). Apoptosis was quantified after 5 days using apoptosis detection kit (BD Biosciences, San Diego, Calif., USA). Staining of cells with the combination of annexin-V-FITC and propidium iodide (PI) was performed according to manufacturer's instructions for detection of live (annexin-V-FITC^{neg}/PI^{neg}) cells, early apoptotic (annexin-V-FITC^{pos}/PI^{neg}) and late apoptotic (annexin-V-FITC^{pos}/PI^{pos}) cells. Specific cell markers were used to examine the apoptotic effect of mAb 146B7 on specific cell populations. NK cells were selected by gating the CD8^{pos}CD56^{pos} cells by flow cytometry (using CD8 specific antibody (clone RPA-T8) and CD56+ (clone B159)); T cells were selected by gating the CD3POS cells (using CD3 specific antibody (cloneUCHT1)); B cells were selected by gating the CD20^{pos} cells by flow cytometry (using CD20+ specific antibody (clone 2H7); all antibodies used for flow cytometry were derived from BD Biosciences). Monocytes were selected based on forward scatter-side scatter (FSC-SSC). Data were analyzed by one-way ANOVA followed by post-hoc Tukey's Multiple Comparison Test. Analysis was performed using Graph Pad Prism (version 3.02 for Windows, Graph Pad Software, San Diego, Calif., USA).

As shown in FIG. 1a-c, mAb 146B7 initiated apoptosis of monocytes, in contrast to a control antibody against KLH. mAb 146B7 increased the fraction of apoptotic monocytes and reduced the fraction of live monocytes following five (5) days of culture in the presence of IL-15. No significant differences in comparison were found in the fraction of live and apoptotic T cells (FIG. 1d-f) and B cells (not shown); a small increase on cell death was seen on NK cells (not shown). It should be noted that the number of T cells in PBMC culture in the presence of IL-15 strongly increased. However, as almost all T cells remained viable in the absence or presence of IL-15, no differences in the fraction of live or apoptotic cells are apparent. For example, 10 μg/ml of mAb 146B7 is shown to inhibit IL-15 mediated survival of about 40% (mean) of monocytes under culture conditions (including 5 ng/ml of IL-15 and 1.5×10⁵ PBMC/well) in which 40% (mean) of the monocyte fraction of PBMC went into late apoptosis during five (5) days of culture. 10 μg/ml of mAb 146B7 is also shown to inhibit IL-15 mediated survival of monocytes by at least about 40-45% (mean), as measured by late apoptosis (vs. control antibody treatment) after five (5) days of culture under culture conditions (including 5 ng/ml of IL-15 and 1.5×10⁵ PBMC/well).

Equivalents

Those skilled in the art will recognize or be able to ascertain, using no more than routine experimentation, many equivalents of the specific embodiments of the invention described herein. Such equivalents are intended to be encompassed by the following claims. Any combination of the embodiments disclosed in the dependent claims are also contemplated to be within the scope of the invention.

Incorporation by Reference

All patents, pending patent applications and other publications cited herein are hereby incorporated by reference in their entirety.

1. A method of treating a disease involving monocytic activity comprising administering a therapeutically effective amount of an IL-15 antagonist that induces apoptosis of monocytes, wherein the disease is gout.

2. A method of treating a disease involving monocytic activity comprising administering a therapeutically effective amount of an IL-15 antagonist that induces apoptosis of monocytes, wherein the disease is a connective disorder selected from the group consisting of systemic sclerosis, retroperitoneal fibrosis, familial Mediterranean fever, and tumor necrosis factor receptor-associated periodic syndromes.

3. A method of treating a disease involving monocytic activity comprising administering a therapeutically effective amount of an IL-15 antagonist that induces apoptosis of monocytes, wherein the disease is a neurological disorder selected from the group consisting of multiple sclerosis, stroke, cerebral trauma, Guillain-Barre syndrome/polyradiculitis, chronic inflammatory demyelinating polyneuropathy, and Alzheimer's disease.

4. A method of treating a disease involving monocytic activity comprising administering a therapeutically effective amount of an IL-15 antagonist that induces apoptosis of monocytes, wherein the disease is a gastrointestinal. or hepatic disorder selected from the group consisting of alcoholic hepatitis, hepatitis C, acute pancreatitis, Whipple's disease, chronic active hepatitis, and sclerosing cholangitis.

5. A method of treating a disease involving monocytic activity comprising administering a therapeutically effective amount of an IL-15 antagonist that induces apoptosis of monocytes, wherein the disease is an allergic disorder selected from the group consisting of chronic urticaria and angioedema.

6. A method of treating a disease involving monocytic activity comprising administering a therapeutically effective amount of an IL-15 antagonist that induces apoptosis of monocytes, wherein the disease is a hematologic disorder selected from the group consisting of hemophagocytic syndrome and histiocytosis X.

7. A method of treating a disease involving monocytic activity comprising administering a therapeutically effective amount of an IL-15 antagonist that induces apoptosis of monocytes, wherein the disease is askin disorder selected from the group consisting of pemphigus vulgaris, toxic/irritative contact eczema, linear IgA dermatitis, dermatitis herpetiformis, epidermolysis bullosa acquisita, acne vulgaris and rosacea.

8. A method of treating a disease involving monocytic activity comprising administering a therapeutically effective amount of an IL-15 antagonist that induces apoptosis of monocytes, wherein the disease is a pulmonary disorder selected from the group consisting of severe acute respiratory distress syndrome, pulmonary silicosis, berylliosis, and asbetosis.

9. A method of treating a disease involving monocytic activity comprising administering a therapeutically effective amount of an IL-15 antagonist that induces apoptosis of monocytes, wherein the disease is is prostatic cancer.

10. A method of treating a disease involving monocytic activity comprising administering a therapeutically effective amount of an IL-15 antagonist that induces apoptosis of monocytes, wherein the disease is an endocrinological disorder selected from the group consisting of insulin-dependent diabetes mellitus, and subacute thyroiditis.

11. A method of treating a disease involving monocytic activity comprising administering a therapeutically effective amount of an IL-15 antagonist that induces apoptosis of monocytes, wherein the disease is a vasculititis selected from the group consisting of panniculitis, erythema nodosum, and Behcet's syndrome.

12. A method of treating a disease involving monocytic activity comprising administering a therapeutically effective amount of an IL-15 antagonist that induces apoptosis of monocytes, wherein the disease is an infectious disorder selected from the group consisting of leishmaniasis and infectious mononucleosis.

13. A method of treating a disease involving monocytic activity comprising administering a therapeutically effective amount of an IL-15 antagonist that induces apoptosis of monocytes, wherein the disease is a kidney disorder selected from the group consisting of chronic renal failure, acute glomerulonephritis, chronic glomerulonephritis, ANCA-associated nephritides, and nephrosclerosis.

14. A method of treating a disease involving monocytic activity comprising administering a therapeutically effective amount of an IL-15 antagonist that induces apoptosis of monocytes, wherein the disease is selected from the group consisting of cardiac disorders, circulatory disorders, metabolic disorders, coagulation disorders, and bone disorders.

15. The method of claim 14, wherein the cardiac disorder is selected from the group consisting of acute myocardial infarction, acute coronary syndromes, and unstable coronary disease.

16. The method of claim 14, wherein the circulatory disorder is selected from the group consisting of arterial hypertension and pulmonary hypertension.

17. The method of claim 14, wherein the metabolic disorder is selected form the group consisting of Gaucher's disease and Fabry's disease.

18. The method of claim 14, wherein the coagulation disorder is selected from the group consisting of disseminated intravascular coagulation, thrombotic thrombocytopenic purpura, and hemolytic-uremic syndrome.

19. The method of claim 14, wherein the bone disorder is osteoporosis.

20. A method of treating a disease comprising administering a therapeutically effective amount of an IL-15 antagonist that induces apoptosis of a cell, wherein the cell is selected from the group consisting of T cells, B cells, neutrophils, mast cells, keratinocytes, NK T cells, and NK cells.

21. The method of claim 1, wherein the antagonist is an agent which binds to IL-15 or IL-15R.

22. The method of claim 1, wherein the antagonist is a human monoclonal antibody that binds to IL-15.

23. The method of claim 1, wherein the antagonist interferes with assembly of the IL-15 receptor α, β, and γ subunits.

24. The method of claim 1, wherein the antagonist binds to an epitope located on the β- or γ-chain interacting domain of IL-15.

25. The method of claim 24, wherein the antagonist specifically binds to an epitope located on the γ-chain interacting domain of human IL-15.

26. The method of claim 24, wherein the antagonist interferes with either the binding of Asp⁸ of human IL-15 to the #3-unit of the human IL-15 receptor or the binding of Gln¹⁰⁸ of human IL-15 to the γ-unit of human IL-15 receptor.

27. The method of claim 22, wherein the antibody comprises at least one CDR sequence selected from the group consisting of:
(i) SEQ ID NOs:5, 6, 7, 8, 9, and 10;
(ii) sequences which have 1 to 3 amino acid deletions, substitutions, or additions compared to SEQ ID NOs:5, 6, 7, 8, 9, and 10; and
(iii) fragments of the sequences defined in (i) or (ii), which retain the ability to specifically bind to human IL-15.

28. The method of claim 22, wherein the antibody comprises a variable heavy chain CDR3 sequence selected from the group consisting of:
(i) SEQ ID NO:7;
(ii) a sequence which has 1 to 3 amino acid deletions, substitutions, or additions compared to SEQ ID NO:7; and
(iii) a fragment of the sequence defined in (i) or (ii), which retains the ability to specifically bind to human IL-15.

29. The method of claim 22, wherein the antibody comprises variable heavy chain CDR1, CDR2 and CDR3 sequences, and variable chain light CDR1, CDR2 and CDR3 sequences, respectively, having the following sequences:
(i) SEQ ID NOs:5, 6, 7, 8, 9, and 10;
(ii) sequences which have 1 to 3 amino acid deletions, substitutions, or additions compared to SEQ ID NOs:5, 6, 7, 8, 9, and 10 (i); or (iii) fragments of the sequences defined in (i) or (ii), which retain the ability to specifically bind to human IL-15.

30. The method of claim 22, wherein the antibody is encoded by human IgG heavy chain and human kappa light chain nucleic acids comprising nucleotide sequences in their variable regions as set forth in FIG. 1 (SEQ ID NO:1) and FIG. 2 (SEQ ID NO:3), respectively, or conservative sequence modifications thereof.

31. The method of claim 22, wherein the antibody has IgG1 heavy chain and kappa light chain variable regions which comprise the amino acid sequences shown in FIG. 1 (SEQ ID NO:2) and FIG. 2 (SEQ ID NO:4), respectively, or conservative sequence modifications thereof.

32. The method of claim 22, wherein the antibody is an IgG1 isotype and has a heavy chain variable region comprising the amino acid sequence shown in FIG. 1 (SEQ ID NO:2), or conservative sequence modifications thereof.

33. The method of claim 22, wherein the antibody is an IgG1 isotype and has a kappa light chain variable region comprising the amino acid sequence shown in FIG. 2 (SEQ ID NO:4), respectively, or conservative sequence modifications thereof.

34. The method of claim 22, wherein the antibody is an antibody fragment or a single chain antibody capable of inducing apoptosis of monocytes.

35. The method of claim 1, wherein the antagonist is a soluble IL-15R.

36. The method of claim 1, wherein the antagonist is an IL-15 mutein.

37. The method of claim 1, wherein treatment of the disease is performed ex vivo.

38. The method of claim 1, wherein treatment of the disease is performed in vivo.


SEQUENCE LISTING (31 sequences)

SEQ ID NO: 1 - DNA, 390 bp, Homo sapiens
  CDS (1)...(390)
gaggtgcagctggtgcagtctggagcagaggtgaaaaagcccggggagtctctgaagatctcctgtaaggtttctggatacttctttaccacctactggatcggctgggtgcgccagatgcccgggaaaggcctggagtatatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagagggggtaactggaactgctttgactactggggccagggaaccctggtcaccgtctcctcagcctccaccaagggcccatcggtcttccccctggca

SEQ ID NO: 2 - protein, 130 aa, Homo sapiens
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLA

SEQ ID NO: 3 - DNA, 357 bp, Homo sapiens
  CDS (1)...(357)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatcccgcagggccactggcatcccagacaggttcagtggcagtgggtctgggacagacttcactctcaccatcagcagactggagcctgaagattttgcagtgtattactgtcagcggtatggtagctcacacacttttggccaggggaccaagctggagatcagccgaactgtggctgcaccatctgtcttcatcttcccg

SEQ ID NO: 4 - protein, 119 aa, Homo sapiens
EIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFP

SEQ ID NO: 5 - protein, 5 aa, Homo sapiens
TYWIG

SEQ ID NO: 6 - protein, 17 aa, Homo sapiens
IIYPGDSDTRYSPSFQG

SEQ ID NO: 7 - protein, 8 aa, Homo sapiens
GNWNCFDY

SEQ ID NO: 8 - protein, 12 aa, Homo sapiens
RASQSVSSSYLA

SEQ ID NO: 9 - protein, 7 aa, Homo sapiens
GASRRAT

SEQ ID NO: 10 - protein, 8 aa, Homo sapiens
QRYGSSHT

SEQ ID NO: 11 - DNA, 20 bp, Homo sapiens
caggtkcagctggtgcagtc

SEQ ID NO: 12 - DNA, 20 bp, Homo sapiens
saggtgcagctgktggagtc

SEQ ID NO: 13 - DNA, 20 bp, Homo sapiens
gaggtgcagctggtgcagtc

SEQ ID NO: 14 - DNA, 20 bp, Homo sapiens
atggactggacctggagcat

SEQ ID NO: 15 - DNA, 21 bp, Homo sapiens
catggaattggggctgagctg

SEQ ID NO: 16 - DNA, 20 bp, Homo sapiens
atggagtttggrctgagctg

SEQ ID NO: 17 - DNA, 21 bp, Homo sapiens
atgaaacacctgtggttcttc

SEQ ID NO: 18 - DNA, 20 bp, Homo sapiens
atggggtcaaccgccatcct

SEQ ID NO: 19 - DNA, 21 bp, Homo sapiens
tgccagggggaagaccgatgg

SEQ ID NO: 20 - DNA, 20 bp, Homo sapiens
racatccagatgayccagtc

SEQ ID NO: 21 - DNA, 20 bp, Homo sapiens
gycatcyrgatgacccagtc

SEQ ID NO: 22 - DNA, 20 bp, Homo sapiens
gatattgtgatgacccagac

SEQ ID NO: 23 - DNA, 20 bp, Homo sapiens
gaaattgtgttgacrcagtc

SEQ ID NO: 24 - DNA, 20 bp, Homo sapiens
gaaatwgtratgacacagtc

SEQ ID NO: 25 - DNA, 20 bp, Homo sapiens
gatgttgtgatgacacagtc

SEQ ID NO: 26 - DNA, 20 bp, Homo sapiens
gaaattgtgctgactcagtc

SEQ ID NO: 27 - DNA, 24 bp, Homo sapiens
cccgctcagctcctggggctcctg

SEQ ID NO: 28 - DNA, 23 bp, Homo sapiens
ccctgctcagctcctggggctgc

SEQ ID NO: 29 - DNA, 26 bp, Homo sapiens
cccagcgcagcttctcttcctcctgc

SEQ ID NO: 30 - DNA, 27 bp, Homo sapiens
atggaaccatggaagccccagcacagc

SEQ ID NO: 31 - DNA, 20 bp, Homo sapiens
cgggaagatgaagacagatg